(19) United States
(12) Patent Application Publication
(10) Pub. No.: US 2007/0089323 A1
(43) Pub. Date: Apr. 26, 2007

(54) Electrothermal massage shoes

(75) Inventor: Ming Lai Wu, Hsin-Tein City (TW)

Correspondence Address: TROXELL LAW OFFICE PLLC, SUITE 1404, 5205 LEESBURG PIKE, FALLS CHURCH, VA, 22041, US

(73) Assignee: Ta Lai Sporting Goods Enterprise Co., Ltd.

(21) Appl. No.: 11/258,047

(22) Filed: Oct. 26, 2005

(51) Int. Cl. A61F 5/14

(52) U.S. Cl. 36/141

(57) ABSTRACT

The present invention provides an electrothermal massage shoes, mainly comprising an electrothermal tube, conductive metal plate and low frequency metal web in the mid sole of shoes like sleepers, outdoors shoes, sports shoes, leisure shoes and hiking shoes, and shoe sole made of moisture-absorbing and sweat-repelling material and equipped with a remote controller that operates at a fixed frequency. Thus, when the user is wearing the shoes, the user's feet can receive thermal therapy due to heat generated by the electrothermal tube and enjoy massage though easy remote control.

### BACKGROUND OF THE INVENTION

1. Field of the Invention

The present invention is related to an improvement on shoes, which can provide therapy and massage. Particularly, the shoes can be adjusted for intensity by users through remote control or cable control to provide the desired intensity for electrothermal therapy and massage at any time in any place. The substantial benefits delivered by the massage shoes include promoting blood circulation and metabolism and stimulating peripheral nerves.

2. Description of the Prior Art

Due to busy life style and stress, more and more civilized diseases are found. Therefore, many rehabilitation equipments specific to certain civilized diseases can also be found in the market. Such rehabilitation equipments designed for specific needs pose difficulty in their manufacturing with high cost. This is the main reason why their market price is high and they do not get popularized.

Besides such rehabilitation equipments with specific purpose are designed for certain groups of patients. Common people would have physiological problems with poor blood circulation and slow metabolism. When the weather gets cooler and exercise activity becomes less, the situation will get worse for people like office workers sitting too long, students sitting in classroom or seniors with movement difficulty. It is even possible to result in sluggish peripheral nerve response. Such a problem could happen to any people. It will not only affect daily life but also become a tough disease.

Usually, it is a relatively long period of time for people wearing shoes every day. The shoes include sleepers, outdoor shoes, sports shoes, leisure shoes and hiking shoes etc. This indicates shoes are in a great deal of use. The present invention is intended to provide a simple and practical means to design rehabilitation equipment suitable for home use and effective treatment and improvement on common physiological problems.

Thus, the inventor with innovative and dedicative spirit invented safe and energy-saving electrothermal massage shoes that can be used by any person at any time in any place.

### SUMMARY OF THE INVENTION

The primary objective for the invention is to provide an innovative electrothermal massage shoes with electrothermal mechanism inside the mid sole, which includes electrothermal tube, conductive metal plate and low frequency therapy area formed in metal web. The shoes use moisture-absorbing and sweat-repelling material for out sole. Besides, the massage shoes are equipped with a remote or cable controller, so the user can adjust the intensity to assure the desired electrothermal therapeutic effect.

Another objective for the invention is to provide an innovative electrothermal massage shoes that can be used as various kinds of shoes, such as sleepers, indoor shoes, outdoor shoes, sports shoes, leisure shoes and hiking shoes etc.

To attain the above objectives, the electrothermal massage shoes for the present invention comprise in structure at least:

A shoe face;

A mid sole, inside which an electrothermal tube is to provide thermal energy for therapy, covered with a layer of conductive metal plate for uniform thermal effect, and which electrothermal tube has two connectors to link to a electrothermal conductive metal plate and to facilitate external power input through a power adaptor, and which conductive metal plate surface is covered with metal web by high pressure molding process, and therefore a low frequency therapeutic area is formed on the mid sole surface for the user to receive electrothermal therapy and massage; and

A shoe sole, made of moisture-absorbing, sweat-repelling and anti-bacteria material, which is capable of releasing water and steam generated after electrothermal therapy and massage and inhibiting bacterial growth.

Thus, a complete set of electrothermal massage shoes is made. Further through a RF remote controller or a cable controller, the electric current intensity can be adjusted to provide the desired massage effect. Besides the controllability, the device is prevented from external interference and restraints at any time in any place.

To further familiarize the examination committee with the features, technical characteristics and benefits for the present invention, detailed description for a preferred embodiment is provided with illustration and diagram as follows:

### DETAILED DESCRIPTION OF THE PREFERRED EMBODIMENT

The electrothermal massage shoes (1) for the present invention is mainly to impart electrothermal massage function to mid sole (20) and moisture-absorbing, sweat-repelling function and anti-bacteria function to shoe sole (30). In addition, it is equipped with a RF remote controller or a cable remote controller, so the user can adjust the electrothermal intensity without restraint at any time in any place and enjoy electrothermal therapy.

At first, please refer to FIG. 1 and FIG. 4. The electrothermal massage shoes (1) can be applied to a variety of shoes, including outdoor shoes, sleepers, sports shoes, leisure shoes and hiking shoes etc.

Please refer to FIG. 2 and FIG. 3. The electrothermal massage shoes (1) are composed of:
a shoe face (10), similar to common shoes to cover the feet;
a mid sole (20), inside which an electrothermal tube (21) is as shown in FIG. 5 and FIG. 6, and which electrothermal tube (21) can adopt one U type covering large area or multiple number of coil type covering small area for each as the heat source of thermal therapy; besides, which electrothermal tube (21) surface is covered with a layer of an electrothermal conductive metal plate (211) to provide uniform thermal effect in the area surrounded by the electrothermal tube (21); further, at a proper position at periphery of the mid sole (20), which electrothermal tube (21) has an extended connector and conductive metal plate (22) (not shown in the figure) to facilitate external power input through a power adaptor; besides, which conductive metal plate (22) is covered with high pressure molded metal web (23) to wrap the above electrothermal tube (21), electrothermal metal plate (211) and conductive metal plate (22) inside the mid sole (20), and thus a low frequency therapeutic area is formed on the mid sole surface for the user to receive electrothermal therapy and massage;

a show sole (30), connected at the bottom of the mid sole (20), made of moisture-absorbing, sweat-repelling and anti-bacteria material, capable of releasing water and steam generated after electrothermal therapy and massage and inhibiting bacterial growth.

The massage shoes are composed of the above components and adjustable by the user for desired electrothermal massage effect.

Both the electrothermal tube (21) inside the mid sole (20) and the metal web (23) covering the surface can be implemented in the mid sole (20) at the same time to provide electrothermal therapy and massage effect. Alternatively, they can be used separately in the mid sole (20) to provide electrothermal therapy and massage effect.

The above metal web (23) is woven by the super fine metal yarn that the inventor developed earlier and is patent protected. The metal yarn component is made of finely ground metal fiber (the metal fiber is very fine and does not break in the grinding process). Then fibers (including natural fiber and synthetic break in the grinding process). Then fibers (including natural fiber and synthetic fiber) are covered with such ultra fine metal yarn with diameter of 0.028˜0.040 mm. Such metal yarn is much stronger than common cotton yarn. The formed object has good abrasion resistance and extended life. Natural fiber provides softness and flexibility. Metal web (23) constructed by such fibers has good electric and thermal conductivity and softness.

Besides, the electrothermal massage shoes (1) have a controller, which type depends on environmental factors and function. For outdoor applications, it is a RF remote controller. For indoor applications, it is a cable remote controller. Optionally, for thermal therapy, it is a cable remote controller; for massage function, it is either a cable remote controller or a wireless remote controller. In such way, the electrothermal massage shoes (1) can be adjusted for electric current intensity and massage function. On the other hand, the function menu offers options of six types and thirty levels of intensity to the user.

Accordingly, for wireless remote control, frequency is determined by the area and is fixed to have the effective remote control distance within 3 meters. In such way, besides the controllability, external interference is also prevented. Thus, the invention can be used at any time in any place without restraint.

The structural features are described in the above. As for practical application, please refer to FIG. 3. When the user is wearing the electrothermal massage shoes (1), the metal connector at the heel area of the mid sole (20) links to external AC power source. Then, depending on the user's need, the mode of electric therapy, thermal therapy or massage is selected through the controller. In such way, the feet of the user can receive electric therapy, thermal therapy or massage in the low frequency area on the mid sole (20) surface. After a certain period of time of therapy, the shoes promote blood circulation in feet, expedite metabolism and stimulate peripheral nerves. The shoes provide effective indoor rehabilitation to people lacking exercise or seniors, even people with movement difficulty. The moisture or steam due to electric therapy or thermal therapy can be successfully released through the moisture-absorbing and sweat-repelling material in shoe sole and provide dryness and comfort.

In addition, the above electrothermal massage shoes (1) can be used independently or with previously developed functional socks, which are made of ultra fine hollow fiber (coolmax) (containing super fine metal yarn) and crab yarn to provide moisture-absorbing, sweat-repelling and anti-bacteria effects. They also use ultra elastic yarn (lycra) to increase elasticity and high tension to eliminate looseness. So the user's feet can be covered completely. Besides, the high-tension coverage also promotes blood circulation and helps relieve aches and sore feeling. The metal yarn also has electrostatic dissipation effect. Therefore, the dual functions from the electrothermal massage shoes (1) and functional socks tend to balance the positive and negative ions in the user's body, improve immunity and promote health. They also possess high abrasion resistance.

The electrothermal massage shoes in the invention is operated on the principle that low current and thermal energy can stimulate the peripheral nerves at feet and the central nerve system. Such delicate interaction can help blood circulation and promote the health of specific organs, as well as modulate endocrine response and balance autonomic nerve. While stimulating reflex zone, the shoes also help eliminate fatigue.

The features and the benefits for the present invention include:
1. In general area, it can help regulate physiological conditions like blood circulation and metabolism and further stimulate peripheral nerves.
2. In cold area, it can prevent frostbite and keep the user warm.
3. It can be selected for massage mode according to the user's preference.
4. It can be used at any time in any place without restraints.
5. It can be used with a variety of shoes. It is small, thin and short, so the manufacturing cost is much lower than general electrothermal rehabilitation equipment.
6. It can be selected for thermal therapy or electric therapy with convenience in operation.

In summary, the electrothermal massage shoes in the present invention has simple structure, ease of use and can promote blood circulation, expedite metabolism and stimulate peripheral nerve response. It can be used at any time in any place without restraints. Therefore, the present invention can meet the requirements of novelty and convenience. Accordingly, the patent application is filed.

Specific Drawing:

(1) The designated diagram is FIG. 3.

(2) The legends for FIG. 3 are described as below:

| | | | |
|---|---|---|---|
| Electrothermal massage shoes | (1) | | |
| Shoe face | (10) | mid sole | (20) |
| Electrothermal tube | (21) | Conductive metal plate | (22) |
| Metal web | (23) | Shoe sole | (30) |

### BRIEF DESCRIPTION OF THE DRAWINGS

FIG. 1 is a preferred embodiment for the present invention.

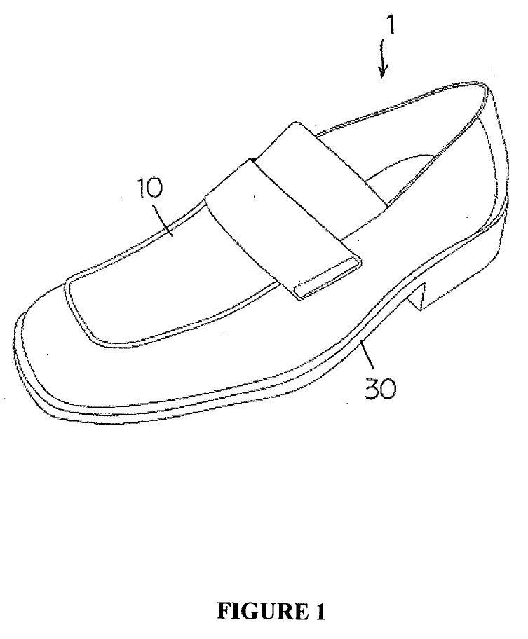

FIG. 2 is the exterior view of another preferred embodiment for the present invention.

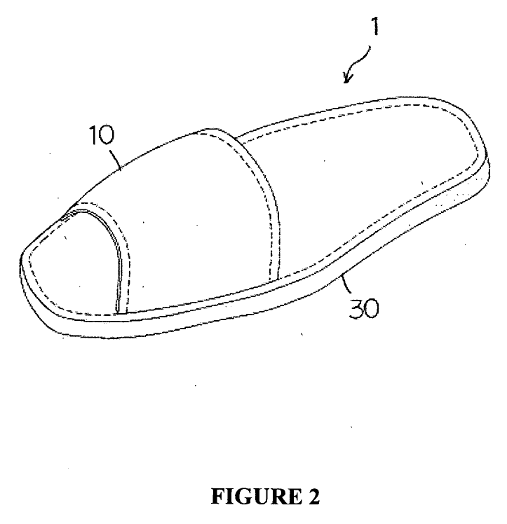

FIG. 3 is the cutaway view for a preferred embodiment for the present invention.

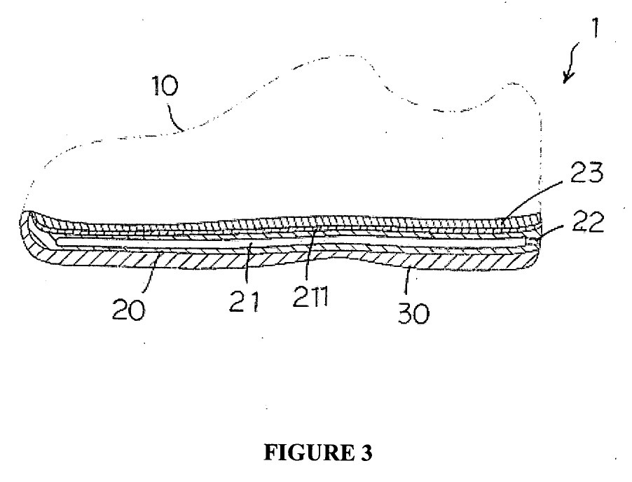

FIG. 4 is the top view for a preferred embodiment for the present invention.

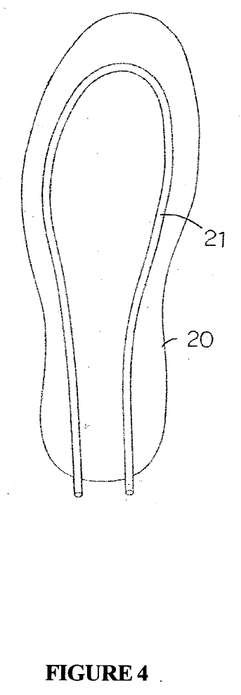

FIG. 5 is the layout for the electrothermal tube inside the mid sole for a preferred embodiment for the present invention.

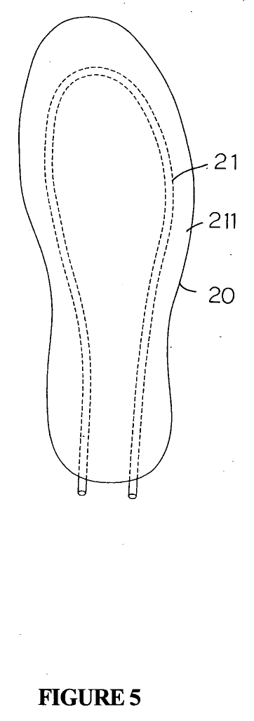

FIG. 6 is the layout for the electrothermal tube inside the mid sole for another preferred embodiment for the present invention.

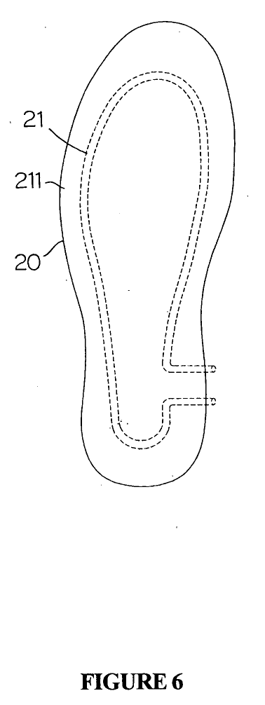

FIG. 7 is the layout for the electrothermal tube inside the mid sole for another preferred embodiment for the present invention.

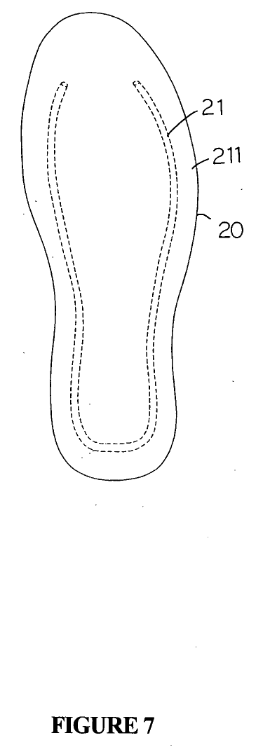

### DESCRIPTION OF MIN COMPONENTS

| | | | |
|---|---|---|---|
| Electrothermal massage shoes | (1) | Shoe face | (10) |
| mid sole | (20) | Electrothermal tube | (21) |
| Electrothermal metal plate | (211) | Conductive metal plate | (22) |
| Metal web | (23) | Shoe sole | (30) |

What is claimed is:

1. An electrothermal massage shoes, comprising:
a shoe face;
a mid sole, inside which is an electrothermal tube, covered with conductive metal plate that has metal web, so the mid sole surface can form a low frequency therapy area; and
a shoe sole, made of moisture-absorbing, sweat-repelling and anti-bacteria material.

2. An electrothermal massage shoes, comprising:
a shoe face;
a mid sole, inside which is an electrothermal tube, covered with conductive metal plate, so the mid sole surface can form a low frequency therapy area; and
a shoe sole made of moisture-absorbing, sweat-repelling and anti-bacteria material.

3. An electrothermal massage shoes, comprising:
a shoe face;
a mid sole, which surface has metal web, so the mid sole surface can form a low frequency therapy area; and
A shoe sole made of moisture-absorbing, sweat-repelling and anti-bacteria material.

4. As described in claim 1 or claim 2 for an electrothermal massage shoes, the electrothermal tube can adopt one U type covering large area inside the mid sole.

5. As described in claim 1 or claim 2 for an electrothermal massage shoes, the electrothermal tube can adopt multiple number of coil type covering small area for each inside the mid sole.

6. As described in claim 1 or claim 3 for an electrothermal massage shoes, the metal web on the mid sole surface is made through integrated high-pressure forming process.

7. As described in claim 1 or claim 2 for an electrothermal massage shoes, the electrothermal tube has two connectors to link to an electrothermal conductive metal plate and to facilitate external power input through a power adaptor.

8. As described in claim 1 or claim 2 for an electrothermal massage shoe, the invention can be applied to various kinds of shoes, such as sleepers, indoor shoes, outdoor shoes, sports shoes, leisure shoes and hiking shoes etc.
